(19) United States
(12) Patent Application Publication
(10) Pub. No.: US 2015/0216709 A1
(43) Pub. Date: Aug. 6, 2015

(54) Nasal Dilator

(71) Applicant: Joe Peck, Portland, OR (US)

(72) Inventor: Joe Peck, Portland, OR (US)

(21) Appl. No.: 14/169,466

(22) Filed: Jan. 31, 2014

(51) Int. Cl. A61F 5/08

(52) U.S. Cl. CPC A61F 5/08

(57) ABSTRACT

A nasal dilator having a constructed truss member consisting of an adhesive backing, an adhesive layer, a resilient layer, and a cover layer coupled to the adhesive layer and whereby the resilient layer is disposed between the cover layer and the adhesive layer.

Any at least one cover, adhesive or backing layer consists of a top edge having a first notch arranged substantially horizontally along the top edge and disposed perpendicular to the common centerline, the first notch intermediate to and connecting to an upwardly and outwardly diverging first-left and first-right portion of the top edge, a bottom edge disposed opposite the top edge, the bottom edge having a second notch arranged substantially horizontally along the bottom edge and disposed perpendicular to the common centerline, the second notch intermediate to and connecting to an downwardly and outwardly diverging second-left and second-right portion of the bottom edge.

### BACKGROUND

The present invention relates to a device and method of dilating external nasal tissue. More specifically, the present invention relates to externally placed nasal dilators having one or more layers, of which one layer is a resilient member.

External devices configured to aid in expanding nasal passages are well-documented in the art. Many such devices include one or more layers that include a resilient truss member configured to pull the external tissue around the nasal passageways in an effort to open the nasal passages. Ierulli in U.S. Pat. No. 8,444,670 issued on May 21, 2013 describes one exemplary device. Therein Ierulli describes a nasal dilator consisting of a laminate of vertical layers to form a truss adapted to engage the outer wall tissues and traverse the bridge of a wearer's nose.

Other examples in the art of external nasal dilators teach a narrower segment configured to bridge the nose and broader portions to arrange on the external tissue above the nasal passages. Some examples of this include U.S. Pat. No. 6,244,265 issued to Cronk et al. on Jun. 12, 2001; U.S. Pat. No. 8,062,329 to Ierulli on Nov. 22, 2011; and U.S. Pat. No. 5,476,091 to Johnson on Dec. 19, 1995, for example.

Yet others teach a deep cleft adjacent to the bridge of the wearer's nose, for example U.S. Pat. No. 8,047,201 issued to Guyuron et al. on Nov. 1, 2011.

And yet others teach a broader segment at the bridge of the wearer's nose. For example, U.S. Pat. No. 8,342,173 to Lockwood, Jr. on Jan. 1, 2013 includes a convex protrusion located near the bridge of the wearer's nose to indicate the proper orientation of the device.

Despite the previous attempts to provide improved breathing from external nasal dilator devices, there remains a need for an improved device that is comfortable to wear, enhances nasal breathing, and improves adhesion to the external tissue while also maximizing the contact area to impart greater outward force on the internal nasal passages to enable reduced restriction of the nasal pathway.

### SUMMARY OF THE INVENTION

The present invention in various preferred embodiments improves upon and otherwise overcomes limitations in the art.

Type I—One preferred embodiment of a nasal dilator for reducing the propensity of nasal passages from contracting during breathing, the dilator comprises: a constructed truss member having a normally substantially planar state, the constructed truss member comprising a plurality of layers, the layers comprising an adhesive backing layer; an adhesive layer removably coupled to the adhesive backing layer; a resilient layer coupled to the adhesive layer; and a cover layer coupled to the adhesive layer whereby the resilient layer is disposed between the cover layer and the adhesive layer. And, further whereby an at least one of the adhesive backing layer, the adhesive layer, or the cover layer is symmetrical with respect to a common centerline and further comprises a top edge having a first notch arranged substantially horizontally along the top edge and disposed perpendicular to the common centerline, the first notch intermediate to and connecting to an upwardly and outwardly diverging first-left and first-right portion of the top edge, a bottom edge disposed opposite the top edge, the bottom edge having a second notch arranged substantially horizontally along the bottom edge and disposed perpendicular to the common centerline, the second notch intermediate to and connecting to an downwardly and outwardly diverging second-left and second-right portion of the bottom edge, and a left and right side portion connecting the top and bottom edges, each left and right side portion comprising at least two arcuate line member segments coupled together, the at least two arcuate line member segments connecting between the top edge and the bottom edge.

Type II—Another preferred embodiment of a nasal dilator, similar to the Type I preferred embodiment, but wherein the resilient layer further comprises a resilient member, the resilient member being symmetrical with respect to a centerline of the resilient member, the resilient member forming a substantially ovoid profile, the ovoid profile being a first height at the centerline and converging to common left side intersection point and a common right side intersection point with respect to the centerline.

Type IIA—Another preferred embodiment alters the Type II embodiment slightly whereby the resilient member further includes an ovoid profile further having a length that is substantially from about three to about four times longer than the first height.

Type IIB—Another preferred embodiment alters the Type II embodiment slightly whereby the resilient member includes an ovoid profile further having a length that is substantially from about four times to about five times longer than the first height.

Layer 2, Type C—The type 1 embodiment is further configured in a new embodiment of nasal dilator wherein the resilient layer further comprises a resilient member, the resilient member being symmetrical with respect to a centerline of the resilient member, the resilient member comprising, with respect to the centerline, a top comprising a top-first side and a top-second side, both the top-first and top-second side diverging upward and outward from the centerline and a top-flat segment configured substantially horizontal and perpendicular to the centerline, the top-flat segment configured intermediate to both the top-first and top-second sides, the resilient member further comprising a bottom disposed opposite from the top, the bottom comprising a bottom-first side and a bottom-second side, both the bottom-first and bottom-second side diverging downward and outward from the centerline and a bottom-flat segment configured substantially horizontal and perpendicular to the centerline, the bottom-flat segment configured intermediate to both the bottom-first and bottom-second sides, the resilient member further comprising a left side extending from the top-first end to the bottom-first end, the left side comprising an arcuate line segment having a first-convex segment adjacent to the top-first side, an intermediate concave segment and an upward-sweeping second-convex segment connecting to the bottom-first side, and the resilient member further comprising a right side extending from the top-second end to the bottom-second end, the right side comprising an arcuate line segment having a first-convex segment adjacent to the top-second side, an intermediate concave segment and an upward-sweeping second-convex segment connecting to the bottom-second side.

LAYERS 1,3, 4 TYPE I—The Type I nasal dilator further configures so that each one of the adhesive backing layer, the adhesive layer, or the cover layer is, individually and respectively, symmetrical with respect to a common centerline and further comprises a top edge having a first notch arranged substantially horizontally along the top edge and disposed perpendicular to the common centerline, the first notch intermediate to and connecting to an upwardly and outwardly diverging first-left and first-right portion of the top edge, a bottom edge disposed opposite the top edge, the bottom edge having a second notch arranged substantially horizontally along the bottom edge and disposed perpendicular to the common centerline, the second notch intermediate to and connecting to an downwardly and outwardly diverging second-left and second-right portion of the bottom edge, and a left and right side portion connecting the top and bottom edges, each left and right side portion comprising at two arcuate line segments connected together to connect to the top edge and bottom edge.

Layers 1, 3, 4—The Type II nasal dilator further configures such that at least one of the adhesive backing layer, the adhesive layer, or the cover layer is symmetrical with respect to a common centerline and further comprises a left side indent and a right side indent each indent respectively disposed on the corresponding left side or right side portion and further wherein the corresponding left side and right side indent is disposed at a base of an intermediate concave section disposed between each one of the at least two arcuate line member segments of the left side portion or right side portion and wherein the at least two arcuate line member segments consists of two concave line arcuate line member segments having an intermediate convex section there-between.

Type III—Yet another embodiment of a nasal dilator includes the Type I dilator modified so that the top edge first notch is an elongated notch; the bottom edge second notch is an elongated notch; and the left and right side portion connecting the top and bottom edges further comprises at least four concave line segment members adjacently arranged and configured to link the top edge and bottom edge.

Type IIIA—The Type III nasal dilator is further adapted whereby the left and right at least four concave line segment members consists of four concave line segment members respectively on the left side portion and the right side portion; a left slot extending horizontal and substantially parallel to the top edge, the left slot disposed on the left side portion between a second and third concave line segment members of the four concave line segment members of the left side portion; and a right slot extending horizontal and substantially parallel to the top edge, the right slot disposed on the right side portion between a second and third concave line segment members of the four concave line segment members of the right side portion.

Type IIIB—The Type III nasal dilator is further adapted whereby the left and right at least four concave line segment members consists of four concave line segment members respectively on the left side portion and the right side portion; a left extended-slot extending horizontal and substantially parallel to the top edge, the left extended-slot disposed on the left side portion between a second and third concave line segment members of the four concave line segment members of the left side portion; and a right extended-slot extending horizontal and substantially parallel to the top edge, the right extended-slot disposed on the right side portion between a second and third concave line segment members of the four concave line segment members of the right side portion.

Type IIIC—The Type III nasal dilator is again adapted whereby the left and right at least four concave line segment members consists of five concave line segment members respectively on the left side portion and the right side portion; the left side portion further comprising a first slot disposed between a second and a third concave line segment member and a second slot disposed between the third concave line segment member and a fourth concave line segment member; and the right side portion further comprising a first slot disposed between a second and a third concave line segment member and a second slot disposed between the third concave line segment member and a fourth concave line segment member.

These aforementioned embodiments, and other embodiments are further described in the detailed description below and those skilled in the art will appreciate the benefits and improvements over the state of the current art.

### DRAWING

FIG. 1 is a top view of a first preferred embodiment according to the present invention.

FIG. 2 is an offset frontal view showing an assembly of components of the embodiment of FIG. 1.

FIG. 3 is a top view of a second preferred embodiment according to the present invention.

FIG. 4 is an offset frontal view showing an assembly of components of the embodiment of FIG. 3.

FIG. 5 is a top view of a third preferred embodiment according to the present invention.

FIG. 6 is an offset frontal view showing an assembly of components of the embodiment of FIG. 5.

FIG. 7 is a top view of a fourth preferred embodiment according to the present invention.

FIG. 8 is an offset frontal view showing an assembly of components of the embodiment of FIG. 7.

FIG. 9 is a top view of a fifth preferred embodiment according to the present invention.

FIG. 10 is an offset frontal view showing an assembly of components of the embodiment of FIG. 9.

FIG. 11 is a top view of a sixth preferred embodiment according to the present invention.

FIG. 12 is an offset frontal view showing an assembly of components of the embodiment of FIG. 11.

FIG. 13 is a top view of an eighth preferred embodiment according to the present invention.

FIG. 14 is a top view of a ninth preferred embodiment according to the present invention.

FIG. 15 is a top view of a tenth preferred embodiment according to the present invention.

FIG. 16 is a top view of another embodiment of the present invention.

FIG. 17 is a top view of another embodiment of the present invention.

FIG. 18 is a top view of another embodiment of the present invention.

FIG. 19 is a top view of another embodiment of the present invention.

FIG. 20 is a top view of another embodiment of the present invention.

FIG. 21 is a top view of another embodiment of the present invention.

FIG. 22 is a top view of another embodiment of the present invention.

FIG. 23 is a top view of another embodiment of the present invention.

FIG. 24 is a top view of another embodiment of the present invention.

FIG. 25 is a top view of another embodiment of the present invention.

FIG. 26 is a top view of another embodiment of the present invention.

FIG. 27 is a top view of another embodiment of the present invention.

FIG. 28 is a top view of another embodiment of the present invention.

FIG. 29 is a top view of another embodiment of the present invention.

FIG. 30 is a top view of another embodiment of the present invention.

FIG. 31 is a top view of another embodiment of the present invention.

FIG. 32 is a top view of another embodiment of the present invention.

FIG. 33 is a top view of another embodiment of the present invention.

FIG. 34 is a top view of another embodiment of the present invention.

FIG. 35 is a top view of another embodiment of the present invention.

FIG. 36 is a top view of another embodiment of the present invention.

FIG. 37 is a top view of another embodiment of the present invention.

FIG. 38 is a top view of another embodiment of the present invention.

FIG. 39 is a top view of another embodiment of the present invention.

FIG. 40 is a top view of another embodiment of the present invention.

FIG. 41 is a top view of another embodiment of the present invention.

FIG. 42 is a top view of another embodiment of the present invention.

FIG. 43 is a top view of another embodiment of the present invention.

FIG. 44 is a top view of yet another embodiment of the present invention.

### DESCRIPTION OF THE INVENTION

Possible preferred embodiments will now be described with reference to the drawings and those skilled in the art will understand that alternative configurations and combinations of components may be substituted without subtracting from the invention. Also, in some figures certain components are omitted to more clearly illustrate the invention.

In the various figures of the drawing some elements are omitted for clarity. In particular, in a given figure, a plurality of similar layers having a similar shape and configuration, a unique reference number does not necessarily reference each individual feature of each layer. Those skilled in the art will appreciate that the similar features share the same reference number and need not be repeatedly called out and otherwise clutter the figure.

In various preferred embodiments of the present invention a nasal dilator consists of a two or three layer structure termed a constructed truss. One layer includes an acrylic based adhesive layer having an adhesive on one side and a resilient member attached to the opposite side. The nasal dilator has moisture resistant properties and can be made from reflective material. One suitable reflective material is a customized coated material that contains a reflective ink. The reflective features will act as both a safety and breathing function for the device. The reflection will occur from approaching light from vehicles, or any other object that shines light in the direction of the nose. It will light up solid colors or imprints including logos. The reflective ink will be applied to the top layer of the material, underneath the flexor.

Another feature of the product is moisture resistance. Any layer, but particularly the adhesive layer is a customized coated material (such as polyethylene) that contains a moisture resistant barrier to prevent liquids from getting underneath the skin-contacting side of the dilator. The barrier will be impervious to sweat, rain, mud or snow, keeping the device in tact and functioning if moisture is applied.

One particular advantage of the preferred embodiments of the present invention using two-layers consisting of an adhesive layer and a resilient layer is the ability of the wearer to better position the dilator in the correct location on the wearer's nose. With the correct positioning, the wearer experiences more comfort over usage time, and the areas of where the lifting action occurs is more clearly identified. Further, the position of the flexor and the flared ends of the material provide better bond to the skin to ensure better hold and performance.

The resilient member in many preferred embodiments is a solid, one-piece member that is designed to improve the user's breathing performance by providing multiple lifting action at five key points around the nose including the bridge, where the flexor band is narrower to lift at the front and limit the pressure on the nose, and at the top and bottom on each side of the nose to lift the narrower regions of the nasal passages. In some embodiments the resilient member band widens on each end to accomplish this, and the impact is evenly distributed. The design also fits more nose shapes and helps keep the strip from lifting up. In contrast, the conventional single or double band systems cause more force on the front of the nose, limit the lifting action, have a tendency to pop off, and are less comfortable. Thus, the solid flexor, combined with the custom adhesives, improve performance in all areas.

### Type I (or II without ‘Indent’ and Generic Type III)

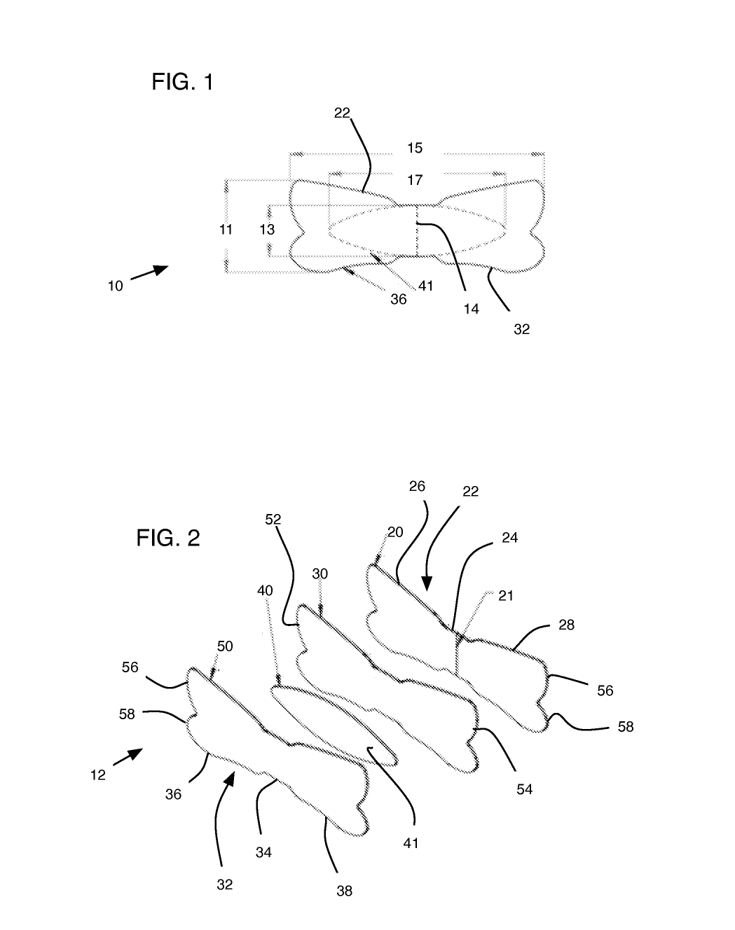

FIGS. 1-15 illustrate various embodiments of an improved nasal dilator 10. Common to these various embodiments, the nasal dilator 10 reduces the propensity of nasal passages from contracting during breathing.

The nasal dilator has an overall height 11, a mid-height 13, an overall length 15 and a resilient member length 17.

The various preferred embodiments commonly include a constructed truss member 12 having a normally substantially planar state and the constructed truss member consists of a plurality of layers, such as at least two, and possibly three to four layers. The layers include an adhesive backing layer 20, an adhesive layer 30 removably coupled to the adhesive backing layer 20, a resilient layer 40 coupled to the adhesive layer, and an optional cover layer 50 coupled to the adhesive layer whereby the resilient layer is disposed the adhesive layer.

Further, any at least one of the plurality of layers (and preferably each of the plurality of layers comprising the adhesive backing layer 20, the adhesive layer 30, or the optional cover layer 50) is symmetrical with respect to a common centerline 14. Any one, or preferably each, of these layers further includes a top edge 22 having a first notch 24 arranged substantially horizontally along the top edge and disposed perpendicular to the common centerline 14. The first notch arranges intermediate to and connecting to an upwardly and outwardly diverging first-left 26 and first-right portion 28 of the top edge.

A bottom edge 32 arranges and is disposed opposite to the top edge 22. The bottom edge has a second notch 34 arranged substantially horizontally along the bottom edge and disposed perpendicular to the common centerline 14. The second notch 34 arranges intermediate to and connecting to a downwardly and outwardly diverging second-left 36 and second-right 38 portion of the bottom edge.

A left 52 and right 54 side portion connect to and otherwise link the top and bottom edges. Each left and right side portion (52 and 54, respectively) include at least two arcuate line member segments 56 58 coupled together. These at least two arcuate line member segments connect between the top edge and the bottom edge.

FIGS. 1 and 2 illustrate a first preferred embodiment according to the present invention. The resilient member layer 40 is an ovoid 41 having a length 17 that is substantially less than the overall length 15 of the dilator 10. The height of the ovoid resilient member 41 is substantially the same as the mid-height 13 of the dilator 10.

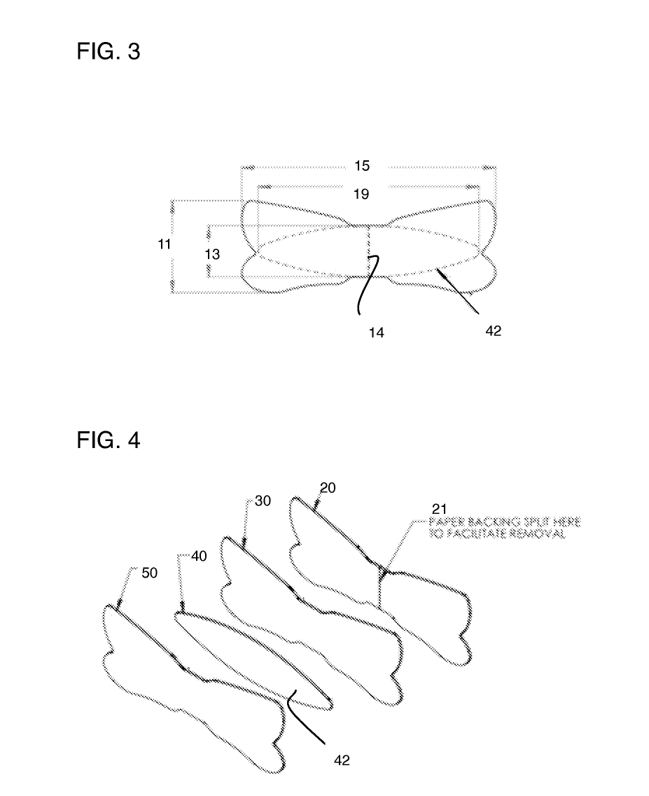

FIGS. 3 and 4 illustrate a second preferred embodiment wherein the adhesive backing layer 20, the adhesive layer 30, and the cover layer 50 are each identical in shape and configuration as described with respect to FIGS. 1 and 2 and the first preferred embodiment. However, in this second preferred embodiment the resilient layer 40 consists of a larger ovoid member 42, which is again symmetrical with respect to a centerline 14. The larger ovoid member 42 has a length 19 that is substantially close to the overall length 15 of the dilator 10. The height of the ovoid resilient member 42 is substantially the same as the mid-height 13 of the dilator 10. Further, the ovoid profile being a first height at the centerline and converging to common left side intersection point and a common right side intersection point with respect to the centerline.

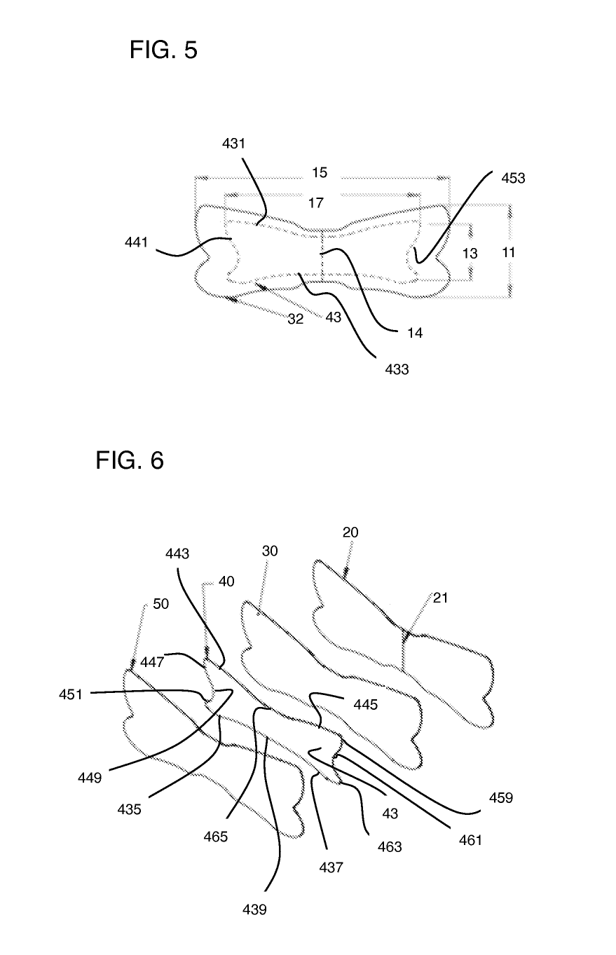

FIGS. 5 and 6 illustrate a third preferred embodiment of the present invention. The backing layer 20, the adhesive layer 30, and the cover layer 50 are similar to the corresponding layers of the first preferred embodiment. Accordingly, the details of these similar layers are omitted in this description of the third embodiment as such discussion is duplicative and would otherwise distract from more salient features of the resilient layer 40.

The resilient member is symmetrical with respect to a vertical centerline of the resilient member. The resilient member comprising, with respect to the centerline, a top 431 comprising a top-first side 443 and a top-second side 445, both the top-first side 443 and top-second side 445 diverging upward and outward from the centerline and a top-flat segment 465 configured substantially horizontal and perpendicular to the centerline, the top-flat segment configured intermediate to both the top-first and top-second sides,

The resilient layer 40 consists of a resilient member 43, which includes a bottom disposed 431 opposite from the top 433. The bottom includes a bottom-first side 435 and a bottom-second side 437. Both the bottom-first and bottom-second side diverge downward and outward from the centerline and a bottom-flat segment 439 arranges and is otherwise configured substantially horizontal and perpendicular to the centerline. The bottom-flat segment 439 configured intermediate to both the bottom-first and bottom-second sides.

The resilient member 43 further includes a left side 441 extending from the top-first side 443 to the bottom-first side 435, the left side 441 comprising an arcuate line segment having a first-convex segment 447 adjacent to the top-first side, an intermediate concave segment 449 and an upward-sweeping second-convex segment 451 connecting to the bottom-first side.

The resilient member 43 further includes a right side 453 extending from the top-second side 455 to the bottom-second side 437. The right side 453 consists of an arcuate line segment having a first-convex segment 459 adjacent to the top-second side, an intermediate concave segment 461 and an upward-sweeping second-convex segment 463 connecting to the bottom-second side.

Each of the aforementioned preferred embodiments illustrated in FIGS. 1-6 include identical layer configurations for the backing layer 20, the adhesive layer 30, and the cover layer 50. Additionally each of these layers consist are symmetrical with respect to a common vertical centerline. And, each layer consists of a top edge 22 having a first notch 24 arranged substantially horizontally along the top edge and disposed perpendicular to the common centerline. The first notch arranges intermediate to and connecting to an upwardly and outwardly diverging first-left 26 and first-right 28 portion of the top edge.

Similarly, a bottom edge 32 is disposed opposite the top edge. The bottom edge having a second notch 34 arranged substantially horizontally along the bottom edge and disposed perpendicular to the common centerline. The second notch arranges intermediate to and connecting to a downwardly and outwardly diverging second-left 36 and second-right 38 portion of the bottom edge. And, a left 52 and right 54 side portion connecting the top and bottom edges. Each left and right side portion comprising at two arcuate line segments 56 58 connected together to connect to the top edge and bottom edge.

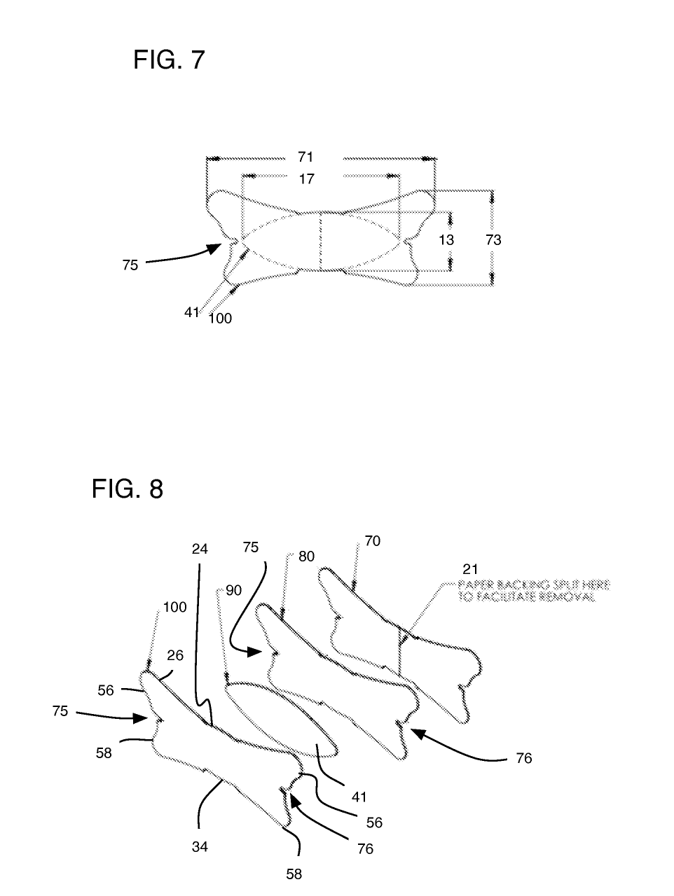

FIGS. 7-12 illustrate another series of similarly configured nasal dilators 10 according to the present invention. Specifically, in each embodiment discussed below in FIGS. 7-12, the backing layer 70, the adhesive layer 80, and the cover layer 100 are similarly shaped. This particular shape is now detailed with specific reference to FIGS. 7 and 8, but each embodiment of FIGS. 7-12 should be understood to be similarly configured.

FIGS. 7 and 8 illustrate a fourth preferred embodiment according to the present invention. One key distinguishing feature of this embodiment is a notch or indent 75 on both the left and right sides of the cover, adhesive, and backing layers.

The nasal dilator 10 of the first embodiment is modified such that at least one of the adhesive backing layer 70, the adhesive layer 80, or the cover layer 100 is symmetrical with respect to a common vertical centerline and further includes a left side indent 75 and a right side indent 76. Each indent respectively disposed on the corresponding left side 52 or right side 54 portion. And, further the corresponding left side 75 and right side indent 76 is disposed at a base of an intermediate concave section disposed between each one of the at least two arcuate line member segments of the left side portion (56 58) or right side portion and wherein the at least two arcuate line member segments consists of two concave line arcuate line member segments having an intermediate convex section there-between.

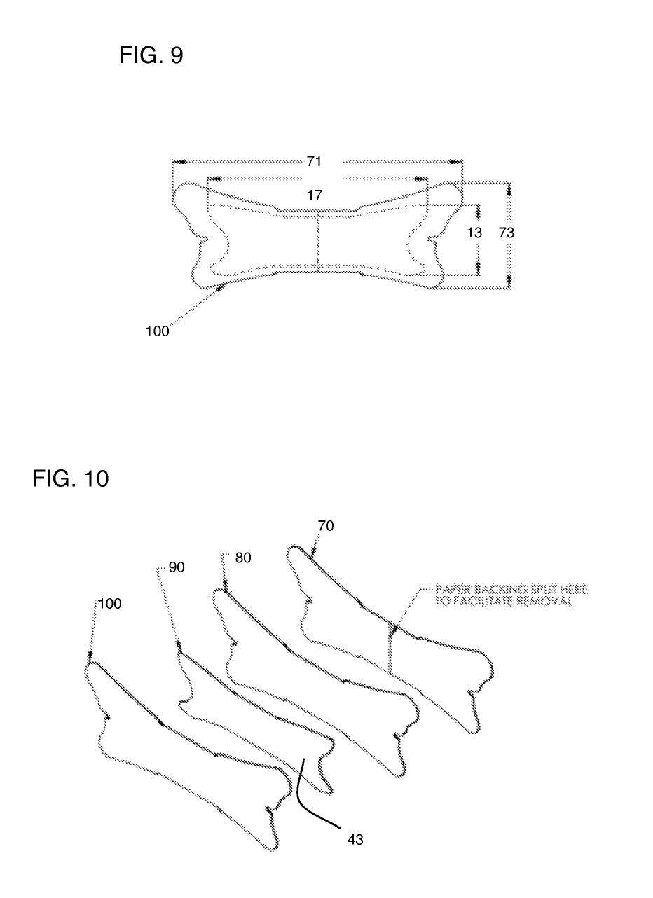

FIGS. 9 and 10 illustrate a fifth preferred embodiment according to the present invention wherein the backing 70, adhesive 80, and cover 100 layers of the embodiment of FIGS. 7 and 8 are combined with the resilient layer 90 consisting of the resilient member 41 of FIGS. 5 and 6.

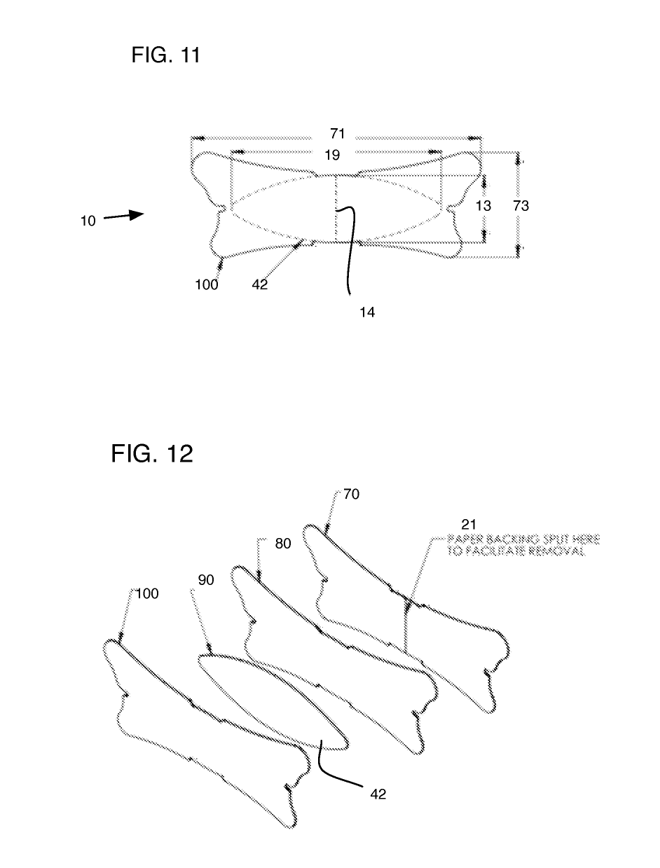

FIGS. 11 and 12 illustrate a sixth preferred embodiment wherein the backing 70, adhesive 80, and cover 100 layers of the embodiment of FIGS. 7 and 8 are combined with the resilient layer 90 consisting of the resilient member 42 of FIGS. 3 and 4.

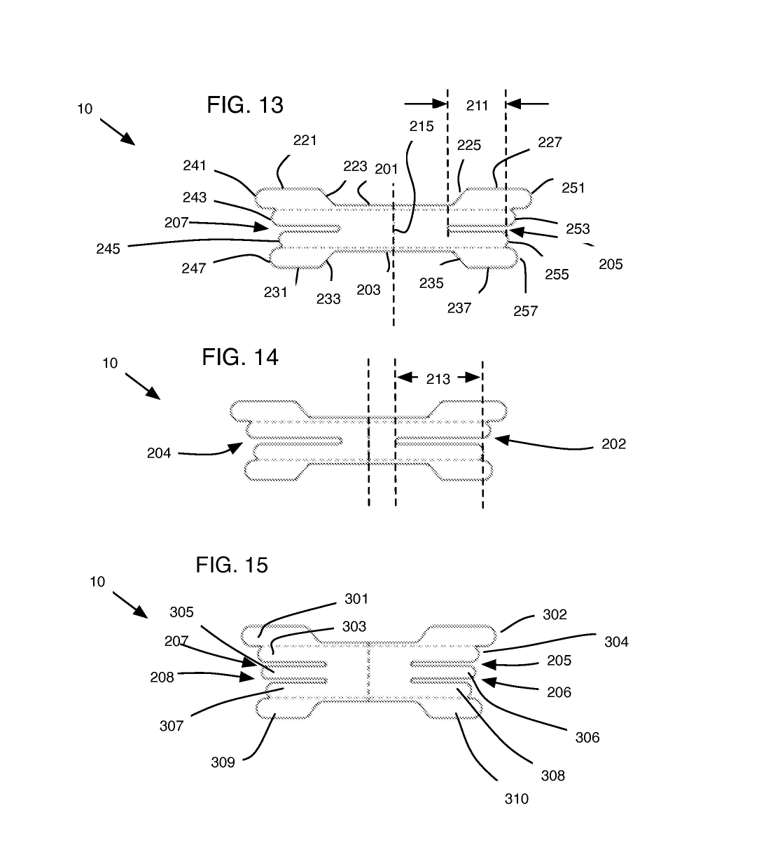

FIGS. 13-15 illustrated yet other preferred embodiments of the present invention. Making specific reference to FIG. 13, this preferred embodiment 10 can be related to the first preferred embodiment with some modifications to certain elements. For example, in the embodiment of FIG. 13 the nasal dilator 10 includes an elongated notch 201 at the top edge first notch and the bottom edge second notch is also an elongated notch 203.

Additionally, the left and right side portion connecting the top and bottom edges further comprises at least four concave line segment members adjacently arranged and configured to link the top edge and bottom edge. For example, on the left side portion the at least four concave line segment member are four concave members 241, 243, 245, and 247, respectively. And, the right side portion includes four concave line segment members 251, 253, 255, and 257, for example.

Further, the embodiment of FIG. 13 also includes a left slot 207 extending horizontal and substantially parallel to the top edge, the left slot disposed on the left side portion between a second 243 and third 245 concave line segment members of the four concave line segment members of the left side portion and the slot has a mid-depth 211. Similarly, a right slot 205 extends horizontal and substantially parallel to the top edge, the right slot disposed on the right side portion between a second 253 and third 255 concave line segment members of the four concave line segment members of the right side portion.

FIG. 14 illustrates yet another preferred embodiment of a nasal dilator 10. Similar to the embodiment of FIG. 13, this embodiment differs by a longer deep slot depth 213, but otherwise is very similar in structure as described above in relation to the embodiment of FIG. 13. Accordingly, the embodiment includes a left extended-slot 204 extending horizontal and substantially parallel to the top edge, the left extended-slot disposed on the left side portion between a second and third concave line segment members of the four concave line segment members of the left side portion and a right extended-slot 202 extending horizontal and substantially parallel to the top edge, the right extended-slot disposed on the right side portion between a second and third concave line segment members of the four concave line segment members of the right side portion.

FIG. 15 illustrates yet another embodiment of a nasal dilator 10. Again, similar to the embodiments of FIGS. 13 and 14, this embodiment, however, includes two slots on both the left and right side. Further, the dilator of this embodiment includes the left and right at least four concave line segment members consists of five concave line segment members (301, 303, 305, 307, and 309) respectively on the left side portion and the right side portion includes members 302, 304, 306, 308, and 310.

Accordingly, the left side portion further comprises a first slot 207 disposed between a second 303 and a third 305 concave line segment member and a second slot 208 disposed between the third 305 concave line segment member and a fourth 307 concave line segment member. And, the right side portion further comprises a first slot 205 disposed between a second 304 and a third 306 concave line segment member and a second slot 206 disposed between the third 306 concave line segment member and a fourth 308 concave line segment member.

In all of the foregoing preferred embodiments of the present invention. The nasal dilator consists of a layered truss. One suitable arrangement of layers includes a first layer consisting of the cover, a second layer consisting of a resilient member, a third layer consisting of an adhesive layer, and a fourth layer consisting of an adhesive backing layer. Other contemplated embodiments include a first layer consisting of a resilient member and a second layer consisting of an adhesive layer.

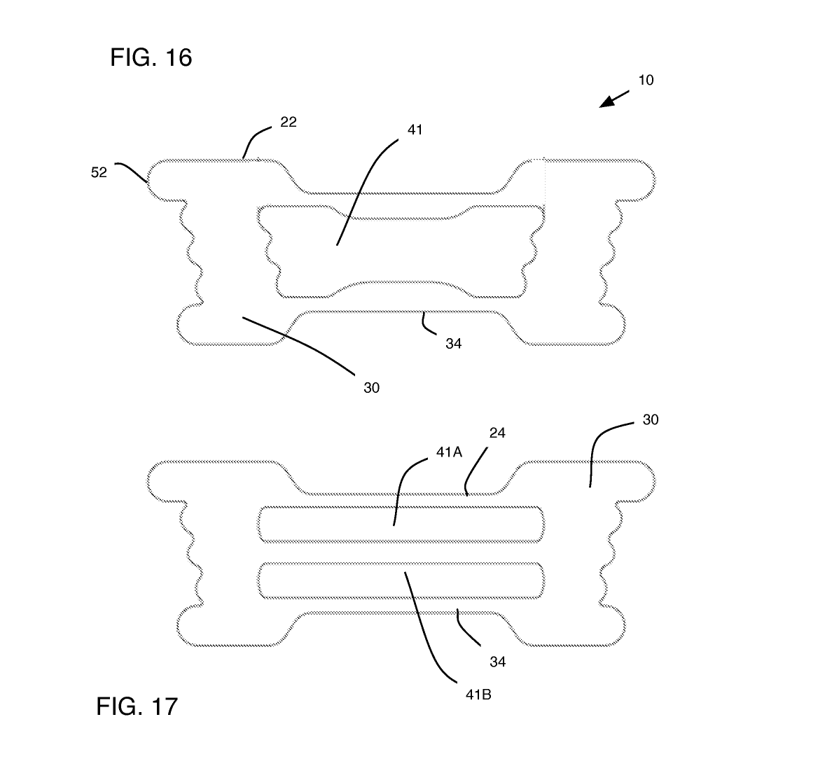

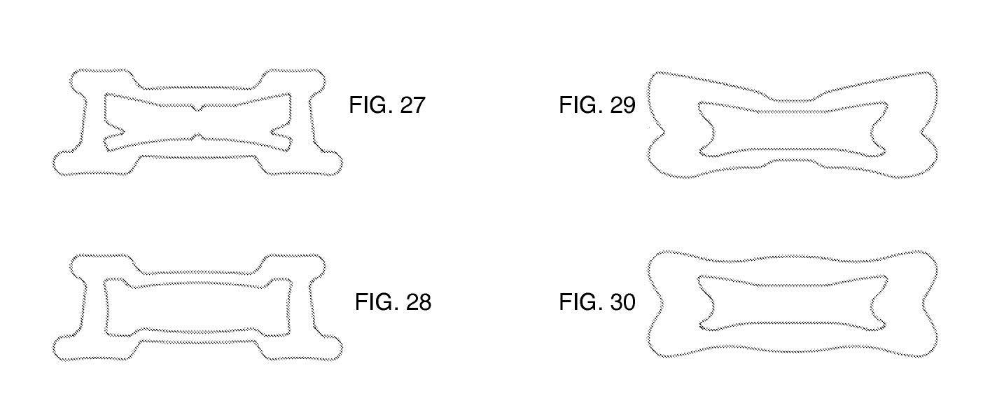

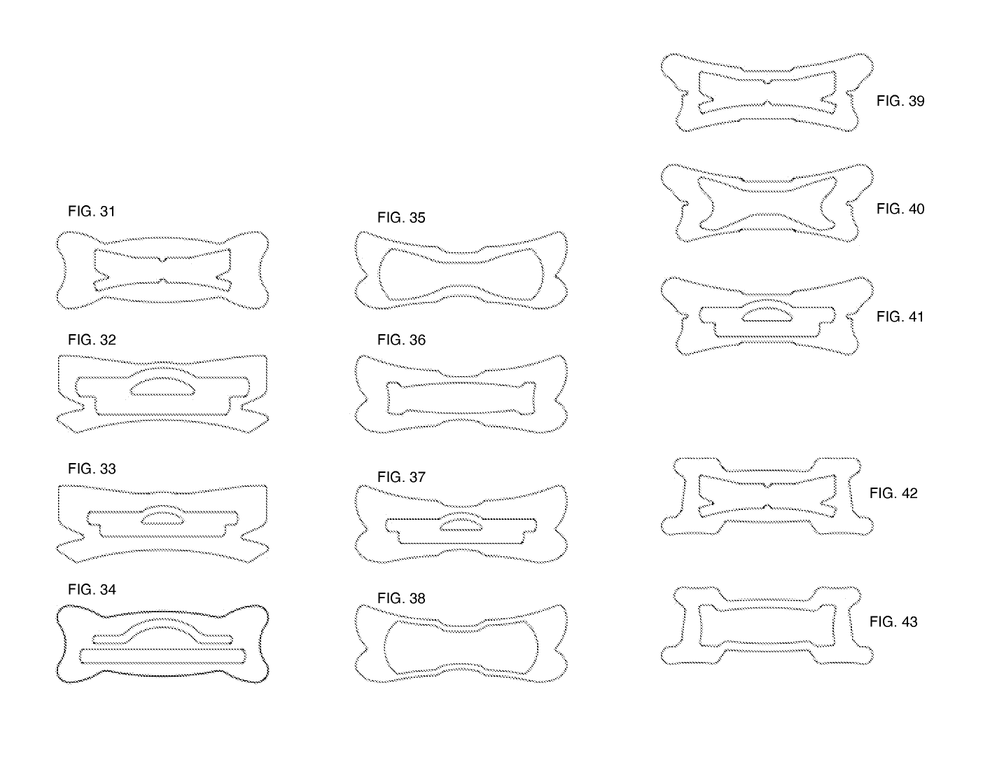

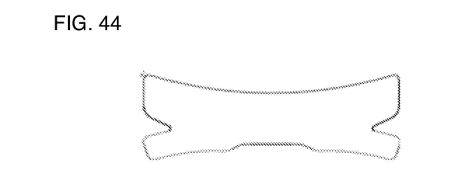

FIGS. 16, and 26-44 illustrate alternative contemplated embodiments of any at least one of the plurality of layers and alternative configurations of any at least one of the one or two resilient members. The Type V dilator comprises a constructed truss member comprising a plurality of layers. The layers comprise an adhesive backing layer, an adhesive layer removably coupled to the adhesive backing layer and a resilient layer coupled to the adhesive layer. And, optionally, a cover layer overlaid on top of the resilient layer. Further, any at least one layer of the plurality of layers is symmetrical with respect to a common centerline and further comprises a top edge having a first notch arranged substantially horizontally along the top edge and disposed perpendicular to the common centerline. The first notch is a concave member on top and is otherwise intermediate to and connecting to an upwardly and outwardly diverging first-left and first-right portion of the top edge. A bottom edge disposed opposite the top edge, the bottom edge having a second notch consisting of a convex member arranged substantially horizontally along the bottom edge and disposed perpendicular to the common centerline, the second notch intermediate to and connecting to an downwardly and outwardly diverging second-left and second-right portion of the bottom edge. And, the top concave notch and the bottom convex notch cooperate to define a substantially bulbous intermediate portion of the dilator. Further, a left and right side portion connect the top and bottom edges, each left and right side portion comprising at least two arcuate line member segments coupled together, the at least two arcuate line member segments connecting between the top edge and the bottom edge layer.

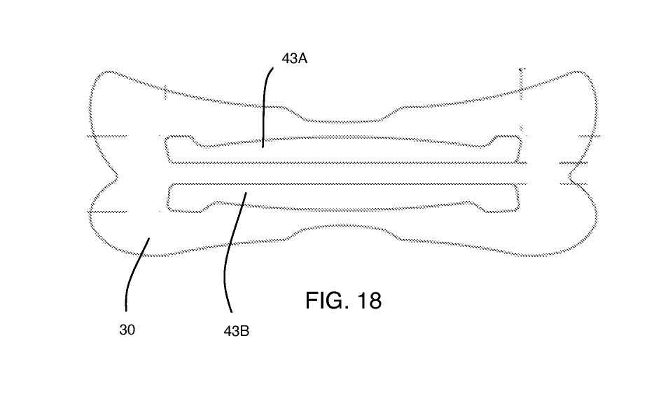

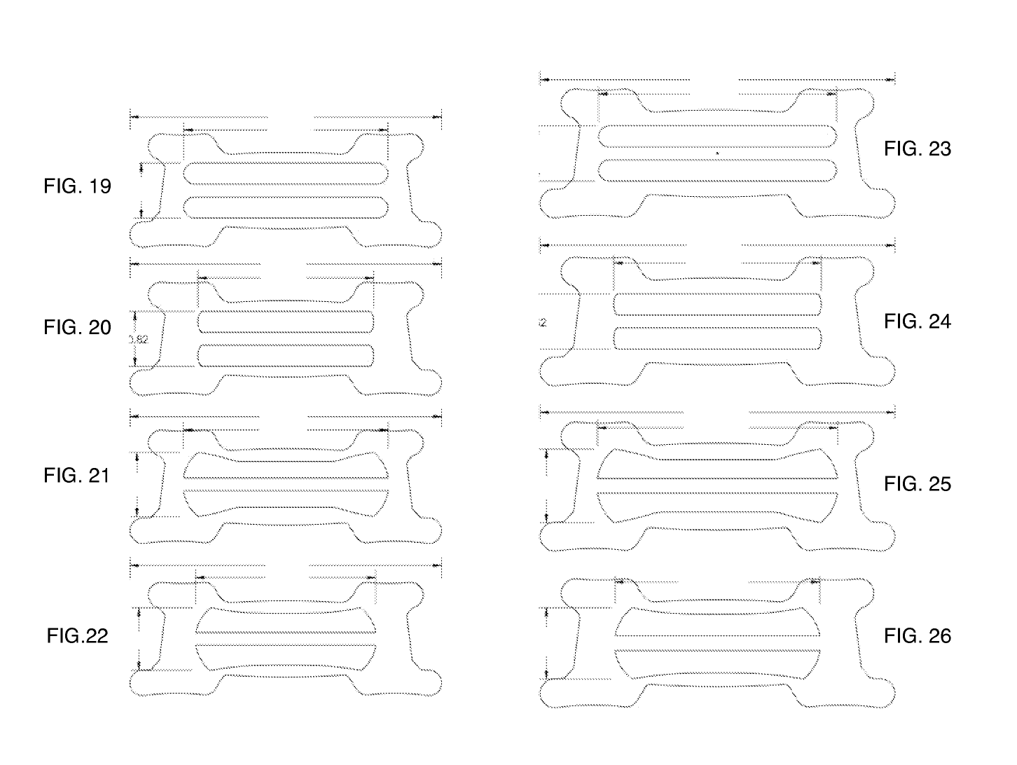

FIGS. 17-25 illustrate another embodiment of the present invention. The nasal dilator includes two resilient members arranged parallel to each other and disposed on the adhesive layer to arrange along a long axis of the dilator. Any of type I, II, Ill, IV or V layers can accommodate two resilient members instead of one.

In the various contemplated and preferred embodiments, the first layer or cover layer can consist of PET (clear or colored, but preferably clear), or a non-woven latex-free material (colored to any desired color or pattern, but preferably to a solid, no-pattern tan). The resilient layer consists of a 10-mil PET flexor. One suitable adhesive is a latex-free adhesive. And, the backing layer is a common paper backing material as would be well understood in the art. Other materials and combinations of materials, including color, texture, and pattern are also contemplated.

Although the invention has been particularly shown and described with reference to certain embodiments, it will be understood by those skilled in the art that various changes in form and detail may be made without departing from the spirit and scope of the invention.

I claim:

1. A nasal dilator for reducing the propensity of nasal passages from contracting during breathing, the dilator comprising:
a constructed truss member having a normally substantially planar state, the constructed truss member comprising a plurality of layers, the layers comprising:
an adhesive layer removably coupled to the adhesive backing layer;
a resilient layer coupled to the adhesive layer; and
further whereby any at least one layer of the plurality of layers is symmetrical with respect to a common centerline and further comprises
a top edge having a first notch arranged substantially horizontally along the top edge and disposed perpendicular to the common centerline, the first notch intermediate to and connecting to an upwardly and outwardly diverging first-left and first-right portion of the top edge,
a bottom edge disposed opposite the top edge, the bottom edge having a second notch arranged substantially horizontally along the bottom edge and disposed perpendicular to the common centerline, the second notch intermediate to and connecting to an downwardly and outwardly diverging second-left and second-right portion of the bottom edge, and
a left and right side portion connecting the top and bottom edges, each left and right side portion comprising at least two arcuate line member segments coupled together, the at least two arcuate line member segments connecting between the top edge and the bottom edge.

2. The nasal dilator of claim 1 wherein the resilient layer further comprises:
at least one resilient member, the at least one resilient member being symmetrical with respect to its centerline, the at least one resilient member forming a substantially ovoid profile, the ovoid profile being a first height at the centerline and converging to common left side intersection point and a common right side intersection point with respect to the centerline.

3. The at least one resilient member of claim 2 further comprising:
the ovoid profile further having a length that is substantially from about three to about four times longer than the first height.

4. The at least one resilient member of claim 2 further comprising:
the ovoid profile further having a length that is substantially from about four time to about five times longer than the first height.

5. The nasal dilator of claim 1 wherein the resilient layer further comprises:
at least one resilient member, the resilient member being symmetrical with respect to its centerline, the resilient member comprising, with respect to the centerline, a top comprising a top-first side and a top-second side, both the top-first and top-second side diverging upward and outward from the centerline and a top-flat segment configured substantially horizontal and perpendicular to the centerline, the top-flat segment configured intermediate to both the top-first and top-second sides,
the at least one resilient member further comprising a bottom disposed opposite from the top, the bottom comprising a bottom-first side and a bottom-second side, both the bottom-first and bottom-second side diverging downward and outward from the centerline and a bottom-flat segment configured substantially horizontal and perpendicular to the centerline, the bottom-flat segment configured intermediate to both the bottom-first and bottom-second sides,
the at least one resilient member further comprising a left side extending from the top-first end to the bottom-first end, the left side comprising an arcuate line segment having a first-convex segment adjacent to the top-first side, an intermediate concave segment and an upward-sweeping second-convex segment connecting to the bottom-first side, and
the at least one resilient member further comprising a right side extending from the top-second end to the bottom-second end, the right side comprising an arcuate line segment having a first-convex segment adjacent to the top-second side, an intermediate concave segment and an upward-sweeping second-convex segment connecting to the bottom-second side.

6. The nasal dilator of claim 1 wherein:
any at least one layer of the plurality of layers is symmetrical with respect to a common centerline and further comprises
a top edge having a first notch arranged substantially horizontally along the top edge and disposed perpendicular to the common centerline, the first notch intermediate to and connecting to an upwardly and outwardly diverging first-left and first-right portion of the top edge,
a bottom edge disposed opposite the top edge, the bottom edge having a second notch arranged substantially horizontally along the bottom edge and disposed perpendicular to the common centerline, the second notch intermediate to and connecting to an downwardly and outwardly diverging second-left and second-right portion of the bottom edge, and
a left and right side portion connecting the top and bottom edges, each left and right side portion comprising at two arcuate line segments connected together to connect to the top edge and bottom edge.

7. The nasal dilator of claim 1 wherein any at least one layer of the plurality of layers is symmetrical with respect to a common vertical centerline and further comprises:
a left side indent and a right side indent each indent respectively disposed on the corresponding left side or right side portion and further wherein the corresponding left side and right side indent is disposed at a base of an intermediate concave section disposed between each one of the at least two arcuate line member segments of the left side portion or right side portion and wherein the at least two arcuate line member segments consists of two concave line arcuate line member segments having an intermediate convex section there-between.

8. The nasal dilator of claim 1 wherein:
the top edge first notch is an elongated notch;
the bottom edge second notch is an elongated notch; and
the left and right side portion connecting the top and bottom edges further comprises at least four concave line segment members adjacently arranged and configured to link the top edge and bottom edge.

9. The nasal dilator of claim 8 further comprising:
the left and right at least four concave line segment members consists of four concave line segment members respectively on the left side portion and the right side portion;
a left slot extending horizontal and substantially parallel to the top edge, the left slot disposed on the left side portion between a second and third concave line segment members of the four concave line segment members of the left side portion; and
a right slot extending horizontal and substantially parallel to the top edge, the right slot disposed on the right side portion between a second and third concave line segment members of the four concave line segment members of the right side portion.

10. The nasal dilator of claim 8 further comprising:
the left and right at least four concave line segment members consists of four concave line segment members respectively on the left side portion and the right side portion;
a left extended-slot extending horizontal and substantially parallel to the top edge, the left extended-slot disposed on the left side portion between a second and third concave line segment members of the four concave line segment members of the left side portion; and
a right extended-slot extending horizontal and substantially parallel to the top edge, the right extended-slot disposed on the right side portion between a second and third concave line segment members of the four concave line segment members of the right side portion.

11. The nasal dilator of claim 8 further comprising:
the left and right at least four concave line segment members consists of five concave line segment members respectively on the left side portion and the right side portion;
the left side portion further comprising a first slot disposed between a second and a third concave line segment member and a second slot disposed between the third concave line segment member and a fourth concave line segment member; and
the right side portion further comprising a first slot disposed between a second and a third concave line segment member and a second slot disposed between the third concave line segment member and a fourth concave line segment member.

12. The nasal dilator of claim 1 further comprising:
a cover layer coupled to the adhesive layer whereby the resilient layer is disposed between the cover layer and the adhesive layer.

13. The nasal dilator of claim 1 wherein the resilient layer comprises:
at least two resilient members arranged parallel to each other along a long axis and coupled to the adhesive layer.

14. The nasal dilator of claim 1 wherein:
the top edge first notch comprises a concave segment;
the bottom edge second notch comprises a convex segment, the convex and concave segments cooperating to create a bulbous center portion of the dilator.

15. The nasal dilator of claim 1 wherein:
the resilient member comprises a solid, one-piece flexor system wherein the wearer experiences multiple lifting action at five key points including the bridge, where the flexor band is narrower to lift at the front and limit the pressure on the nose, and at the top and bottom on each side of the nose to lift the narrower regions of the nasal passages.

16. The nasal dilator of claim 1 wherein any at least one layer of the plurality of layers comprises:
a reflective ink coupled to the layer to provide reflective properties to visible light.

17. The nasal dilator of claim 1 wherein any at least one of the plurality of layers comprises:
a coating consisting of polyethylene applied to a surface of the corresponding layer.

18. The nasal dilator of claim 1 wherein the plurality of layers further comprises:
an adhesive backing layer coupled to the adhesive layer on a side opposite from the resilient layer.
